(19) United States
(12) Patent Application Publication
(10) Pub. No.: US 2025/0281455 A1
(43) Pub. Date: Sep. 11, 2025

(54) COMPOSITIONS AND METHODS FOR NEUROABLATION

(71) Applicant: Board of Regents of the University of Nebraska, Lincoln, NE (US)

(72) Inventors: Hanjun Wang, Omaha, NE (US); Steven Lisco, Omaha, NE (US); Irving Zucker, Omaha, NE (US); Thomas Nicholas, Colorado Springs, CO (US); Robert Lobato, Omaha, NE (US); Bin Duan, Omaha, NE (US)

(21) Appl. No.: 19/073,136

(22) Filed: Mar. 7, 2025

Related U.S. Application Data
(60) Provisional application No. 63/562,307, filed on Mar. 7, 2024.

(51) Int. Cl. A61K 31/4025; A61K 9/16; A61P 25/00

(52) U.S. Cl. CPC A61K 31/4025; A61K 9/1647; A61K 9/1652; A61P 25/00

(57) ABSTRACT

Compositions and methods for neuroablation and the treatment diseases or disorders associated with neuronal dysfunction are provided.

This application claims priority under 35 U.S.C. § 119 (e) to U.S. Provisional Patent Application No. 63/562,307, filed on Mar. 7, 2024. The foregoing application is incorporated by reference herein.

### FIELD OF THE INVENTION

This invention relates generally to the field of neuronal diseases and disorders. Specifically, the invention provides compositions and methods for neuroablation and the treatment of diseases or disorders associated with neuronal dysfunction.

### BACKGROUND OF THE INVENTION

Sensory (afferent) neurons innervating peripheral organs enter the central nervous system by one of two routes: 1) through the vagus nerve into the brain stem (medulla) with cell bodies residing in the nodose ganglia and 2) directly into the spinal cord where cell bodies reside in the dorsal root ganglia (DRG). The primary role of these sensory ganglia is to detect local environmental changes in these peripheral organs and to communicate this information to the brain. Central processing of afferent information is integrated and regulates peripheral autonomic (efferent; sympathetic and parasympathetic) ganglia to affect peripheral organs through motor projections in the vagus nerves and paravertebral sympathetic ganglia. Therefore, these peripheral ganglia (both afferent and efferent) play an important role in regulating peripheral organ function including nociceptive perception, cardiovascular function and respiratory function. Abnormal ganglionic neuronal dysfunction can result in a variety of diseases and disorders including chronic pain and cardiopulmonary diseases. Improved therapies for treating abnormal ganglionic neuronal dysfunction and the symptoms associated therewith are needed.

### SUMMARY OF THE INVENTION

In accordance with the instant invention, methods of inhibiting, treating, and/or preventing a disease or disorder are provided. The present invention also provides methods of neuroablation. The methods comprise administering an anti-angiogenesis therapeutic to a subject in need thereof. In certain embodiments, the methods comprise administering an anti-angiogenesis therapeutic to or near a ganglion. In certain embodiments, the anti-angiogenesis therapeutic is an anti-vascular endothelial growth factor (VEGF) therapeutic, an anti-VEGF receptor therapeutic, or an mTOR inhibitor. In certain embodiments, the disease or disorder is caused by neural dysfunction. In certain embodiments, the disease or disorder is caused by ganglionic neuronal dysfunction. In certain embodiments, the disease or disorder is selected from the group consisting of arrythmia, drug resistant arrythmia, heart failure, hypertension, cardiopulmonary disease, acute respiratory distress syndrome, acute lung injury, multi organ failure, and pain such as chronic pain, pain associated with osteoarthritis, arthritis, rheumatoid arthritis, back pain, cancer pain, pain related to peripheral artery disease, pain related to inflammatory conditions, pain associated with pancreatitis, or pain associated with colitis. In certain embodiments, the disease or disorder is arrhythmia, chronic pain, or acute lung injury. In certain embodiments, the anti-angiogenesis therapeutic is administered by a route selected from the group consisting of intra-stellate injection, intra-dorsal root ganglion (DRG) injection, intra-thecal injection, intra-spinal injection, epidural injection, intra-nodose injection, peri-nodose injection, intra-trigeminal ganglia injection, peri-trigeminal ganglia injection, intra-celiac ganglia injection, peri-celiac ganglia injection, intra-superior mesenteric ganglia injection, peri-superior mesenteric ganglia injection, peri-stellate administration, and peri-DRG administration. In certain embodiments, the anti-angiogenesis therapeutic is administered to or near a peripheral ganglion or administered to or near a stellate ganglion.

### BRIEF DESCRIPTIONS OF THE DRAWING

FIGS. 1A and 1B show microvascular structure in stellate ganglia (SG) and dorsal root ganglia (DRG). FIG. 1A provides representative scanning electron microscopy (SEM) images showing the microvascular structures within DRG and SG. Arrows point to the vessels in the DRG and SG. FIG. 1B provides representative 3-D images showing the microvascular structure (GFP staining) in the SG by using a Rosa-tdTomato flox/flox::Tie2 Cre reporter mouse model in combination with the tissue clearance technique.

FIGS. 2A-2E show a hydrogel and microsphere-based delivery system loaded with the anti-VEGF medicine sunitinib (SU). FIG. 2A provides a schematic of the fabrication of SU loaded hydrogels. SU was encapsulated within poly(lactic-co-glycolic) acid (PLGA) microspheres and then further loaded within modified hyaluronic acid (HA) hydrogels. FIG. 2B provides SEM image of SU loaded PLGA microspheres. FIG. 2C shows injectability of SU loaded hydrogels. FIG. 2D provides a graph of the rheological test. FIG. 2E provides a graph of the in vitro release of SU from PLGA microsphere loaded hydrogels (n=3).

FIGS. 3A-3D show the in vitro efficacy validation of the sunitinib delivery system. Specifically, an in vitro evaluation is provided of released SU on HUVEC viability and tube formation. FIG. 3A provides representative images showing the impairment of tube formation after being treated with released SU from hydrogel at day 1-7 (D1-7) and day 21-28 (D21-28). FIG. 3B provides a graph of the MTT assay for cell viability. FIGS. 3C and 3D provide graphs of the semi-quantitative analysis of the amount of branch points and the total tube length based on the images. n=3-5, *p<0.05, **p<0.01, ***p<0.001.

FIG. 4 shows the in vivo efficacy validation of the sunitinib delivery system in the rat stellate ganglia by using immunofluorescence (IF) staining. IF staining of CD31 (a vascular endothelial marker) in the rat SG with injection of hydrogels (control) and SU loaded hydrogels 10 days post SG injection.

FIG. 5 shows the in vivo functional efficacy validation of the sunitinib delivery system in the rat stellate ganglia. Representative tracing showing heart rate (HR) and arterial blood pressure (ABP) responses to electrical stimulation of decentralized SG in a control- or SU loaded hydrogel-treated rat.

### DETAILED DESCRIPTION OF THE INVENTION

The present invention describes novel methods for neuroablation of both sensory and autonomic ganglia and methods of treatment by blocking angiogenesis and thereby inducing neuronal apoptosis and cell death. Specifically, the methods relate to localized administration of an anti-angiogenesis therapy to the desired ganglia.

In accordance with the instant invention, methods of neuroablation are provided. In certain embodiments, the method comprises administering an anti-angiogenesis therapeutic to or around a nerve or ganglion. The administration of the anti-angiogenesis therapeutic results in cell death within the ganglion. For example, administration of the anti-angiogenesis therapeutic results in the ablation and/or death of nerve cell bodies or neurons within the ganglion. In certain embodiments, the method of the instant invention replaces surgical stellate ganglionectomy and other non-specific chemo-neuroablative therapies.

In accordance with the instant invention, methods of inhibiting, treating, and/or preventing a disease or disorder are provided. In certain embodiments, the method comprises administering an anti-angiogenesis therapeutic to or around a nerve or ganglion. The administration of the anti-angiogenesis therapeutic results in cell death within the ganglion. For example, administration of the anti-angiogenesis therapeutic results in the ablation and/or death of nerve cell bodies or neurons within the ganglion. In certain embodiments, the method of the instant invention replaces surgical stellate ganglionectomy and other non-specific chemo-neuroablative therapies.

In certain embodiments, the disease or disorder is caused by neuronal dysfunction. In certain embodiments, the disease or disorder is caused by ganglionic neuronal dysfunction. Examples of diseases and disorders caused by neural dysfunction include, without limitation: arrythmias (including drug resistant arrythmias), heart failure (e.g., chronic heart failure (CHF)), hypertension, cardiopulmonary diseases and disorders (e.g., acute respiratory distress syndrome, acute lung injury), multi organ failure, and pain (e.g., chronic pain, pain associated with osteoarthritis, arthritis, rheumatoid arthritis, back pain, cancer pain, pain related to peripheral artery disease, and pain related to inflammatory conditions such as pancreatitis and colitis).

Methods of inhibiting, treating, and/or preventing cardiac arrhythmia, particularly drug-resistant cardiac arrhythmia, are encompassed by the instant invention. The method comprises administering an anti-angiogenesis therapeutic to a subject in need thereof. In certain embodiments, the method comprises administering an anti-angiogenesis therapeutic to or around a ganglion. In certain embodiments, the method comprises administering an anti-angiogenesis therapeutic to or around a stellate ganglion. In certain embodiments, the method comprises administering an anti-angiogenesis therapeutic by epidural injection. In certain embodiments, the method comprises administering an anti-angiogenesis therapeutic to or around a peripheral ganglion. In certain embodiments, the method comprises administering an anti-angiogenesis therapeutic to or around a thoracic dorsal root ganglion.

For sympathetically mediated arrhythmias, the traditional stellate ganglia block strategy only has a short-term (i.e., lasting a few hours to one day) effect in controlling cardiac arrhythmias by temporarily blocking stellate neuronal activity with local anesthetics. Once the anesthetic effect is withdrawn, cardiac arrhythmias reoccur. Therefore, some drug-resistant cardiac arrhythmia patients receive partial stellate ganglionectomy surgery to permanently remove part of the stellate ganglia. Although this surgical removal procedure can efficiently reduce cardiac arrhythmia, it is not highly recommended by anesthesiologists and cardiologists because of its invasive procedure and side effects. The present invention overcomes these shortcomings of current treatments.

Methods of inhibiting, treating, and/or preventing chronic pain, particularly opioid-free chronic pain, are encompassed by the instant invention. The method comprises administering an anti-angiogenesis therapeutic to a subject in need thereof. In certain embodiments, the method comprises administering an anti-angiogenesis therapeutic to or around a ganglion, such as a ganglion near or closest to the chronic pain. In certain embodiments, the method comprises administering an anti-angiogenesis therapeutic to or around a stellate ganglion. In certain embodiments, the method comprises administering an anti-angiogenesis therapeutic by epidural injection. In certain embodiments, the method comprises administering an anti-angiogenesis therapeutic to a ganglion, such as a ganglion near or closest to the chronic pain, via ultrasound-guided nerve block. In certain embodiments, the method comprises administering an anti-angiogenesis therapeutic to or around a peripheral ganglion. In certain embodiments, the method comprises administering an anti-angiogenesis therapeutic to or around a thoracic dorsal root ganglion.

Methods of inhibiting, treating, and/or preventing lung injury, particularly acute lung injury, are encompassed by the instant invention. In certain embodiments, the method inhibits, treats, and/or prevents acute lung injury-associated multi-organ failure. The method comprises administering an anti-angiogenesis therapeutic to a subject in need thereof. In certain embodiments, the method comprises administering an anti-angiogenesis therapeutic to or around a ganglion. In certain embodiments, the method comprises administering an anti-angiogenesis therapeutic to or around a stellate ganglion. In certain embodiments, the method comprises administering an anti-angiogenesis therapeutic by epidural injection. In certain embodiments, the method comprises administering an anti-angiogenesis therapeutic to or around a peripheral ganglion. In certain embodiments, the method comprises administering an anti-angiogenesis therapeutic to or around a thoracic dorsal root ganglion.

Anti-angiogenic therapeutics can also be referred to angiogenesis inhibitors. Anti-angiogenic therapeutics inhibit the formation and/or growth of blood vessels. Anti-angiogenic therapeutics include but are not limited to small molecules, antibodies, peptides, proteins, antibody fragments, and nucleic acid based therapeutics (e.g., siRNA, miRNA, shRNA, and mRNA-based therapeutics). In certain embodiments, the anti-angiogenesis therapeutic is a small molecule. Specific examples of anti-angiogenic therapeutics include, without limitation:
anti-VEGF therapeutics such as anti-vascular endothelial growth factor (VEGF) antibodies (e.g., anti-VEGF-A) or antigen-binding fragments thereof. Examples of anti-VGF therapeutics include, without limitation:
   bevacizumab (Avastin®),
   ranibizumab (Lucentis®),
   faricimab (Vabysmo®),
   brolucizumab (Beovu®), and
   pegaptanib (Macugen®);
anti-VEGF receptor therapeutics such as inhibitors of VEGF receptors (e.g., VEGF receptor 1, 2, and/or 3, particularly VEGF receptor 2). The anti-VEGF receptor therapeutic may be an anti-VEGF receptor antibody or antigen-binding fragment thereof (e.g., anti-VEGF receptor 1, 2, and/or 3, particularly anti-VEGF receptor 2). The anti-VEGF receptor therapeutic may be a tyrosine-kinase inhibitor (TKI) or a receptor tyrosine kinase (RTK) inhibitor (e.g., a small molecule inhibitor). The therapeutics may also target VEGF signaling pathways through VEGF receptors. Examples of anti-VEGF receptor therapeutics include, without limitation:
   aflibercept (Eylea®, Pavblu™),
   axitinib (Inlyta®),
   cabozantinib (Cometriq® and Cabometyx®),
   lenvatinib (Lenvima®),
   pazopanib (Votrient®),
   ponatinib (Iclusig®),
   ramucirumab (Cyramza®),
   regorafenib (Stivarga®),
   sorafenib (Nexavar®),
   sunitinib (Sutent®),
   vandetanib (Caprelsa®),
   icrucumab (IMC-18F1), and
   nintedanib (Ofev® and Vargatef®);
mTOR inhibitors. mTOR inhibitors may interfere with the synthesis of VEGF. Examples of mTOR inhibitors include, without limitation: everlolimus (Afinitor® or Zortress®) and temsirolimus (Torisel®); and
other therapeutics with anti-angiogenesis activity such as lenalidomide (Revlimid®) and thalidomide (Thalomid®), endostatin, angiostatin, thrombospondin (e.g., thrombospondin 1), itraconazole, and prolactin (e.g., an antiangiogenic peptide of prolactin).

In certain embodiments, the anti-angiogenesis therapeutic is an anti-VEGF receptor therapeutic. In certain embodiments, the anti-angiogenesis therapeutic is a receptor tyrosine kinase (RTK) inhibitor. In certain embodiments, the anti-angiogenesis therapeutic is a small molecule. In certain embodiments, the anti-angiogenesis therapeutic is sunitinib.

The anti-angiogenesis therapeutic may be contained within a composition with a carrier, particularly a pharmaceutically acceptable carrier. The anti-angiogenesis therapeutic may be administered as the compound itself or in a composition and/or as a formulation of the compound such as a prodrug or a hydrogel, liposome, micelle, microparticle formulation, and/or nanoparticle formulation. The formulations may be slow and/or fast release formulations. The compositions may further comprise at least one other therapeutic agent for the treatment of the disease or disorder.

In certain embodiments, the anti-angiogenesis therapeutic is contained in a slow-release formulation. In certain embodiments, the anti-angiogenesis therapeutic is contained within a hydrogel. In certain embodiments, the hydrogel is a macromolecular polymer gel including a network. In certain embodiments, the hydrogel is a polymer matrix able to retain water in a swollen state. In certain embodiments, the hydrogel is crosslinked. In certain embodiments, the hydrogel comprises thiol groups (e.g., functionalized with thiol groups) which form disulfide bonds. Examples of hydrogels include, without limitation, one or more of: gelatin, alginate, chitosan, collagen, silk, fibrin, agarose, chondroitin, elastin, starch, pectin, cellulose, methylcellulose, polyethylene glycol (PEG), polyvinyl alcohol (PVA), sodium polyacrylate, polyacrylamide, starch-acrylonitrile co-polymers, a proteoglycan, elastin, and/or a glycosaminoglycan (e.g., hyaluronic acid, heparin, chondroitin sulfate, or keratan sulfate), other natural or synthetic hydrogels, and derivatives thereof (e.g., del Valle et al., Gels (2017) 3:27). In certain embodiments, the hydrogel is selected from the group consisting of alginate, chitosan, hyaluronic acid, gelatin, silk, fibrin, collagen, elastin, cellulose, agarose, chondroitin, PEG, PVA, and polyacrylamide. In certain embodiments, the hydrogel is biocompatible. In certain embodiments, the hydrogel is biodegradable. In certain embodiments, the hydrogel is non-biodegradable. In certain embodiments, the hydrogel comprises hyaluronic acid. In certain embodiments, the hydrogel comprises thiolated hyaluronic acid. The hydrogel may comprise a microsphere or microparticle such as a polymer microsphere or microparticle.

In certain embodiments, the anti-angiogenesis therapeutic is contained within a polymer microsphere or microparticle. In certain embodiments, the microsphere or microparticle is contained within a hydrogel. Microspheres or microparticles may have an average diameter of about 0.5 μm to about 10 μm, about 1 μm to about 5 μm, or about 1 μm to about 3 μm. The polymer may comprise any biocompatible polymer. The polymer may be biodegradable or non-biodegradable. The polymer may by hydrophobic, hydrophilic, or amphiphilic. In certain embodiments, the polymer is hydrophobic. The polymers of the instant invention may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 10,000, about 2 to about 1000, about 2 to about 500, about 2 to about 250, or about 2 to about 100 repeating units or monomers. The polymers of the instant invention may comprise capping termini. In certain embodiments, the polymer comprises poly(lactic-co-glycolic acid) (PLGA).

The methods of the instant invention may further comprise the administration (sequentially (e.g., before and/or after) and/or simultaneously) of at least one other therapeutic for the treatment of the disease or disorder.

The methods of the instant invention may further comprise diagnosing the disease or disorder in the subject prior to administration of the therapeutic agents of the instant invention.

The anti-angiogenesis therapeutic of the present invention can be administered by any suitable route, for example, by injection (e.g., for local, direct, or systemic administration), oral, pulmonary, topical, nasal or other modes of administration. The anti-angiogenesis therapeutic may be contained within a composition with at least one pharmaceutically acceptable carrier. The anti-angiogenesis therapeutic may be administered by any suitable means including, for example: by injection or by parenteral, intramuscular, intravenous, intraarterial, intraperitoneal, subcutaneous, oral, topical, inhalatory, transdermal, intrapulmonary, intraareterial, intrarectal, intramuscular, intranasal, intrathecal, epidural, intraganglionic, and intra-spinal administration. In certain embodiments, the anti-angiogenesis therapeutic is administered by oral administration, intravenous injection, intrathecal administration, epidural administration, intra-dorsal root ganglia (DRG) administration, or intraganglionic administration. In certain embodiments, the anti-angiogenesis therapeutic is administered by direct injection. In certain embodiments, the anti-angiogenesis therapeutic is administered by direct injection to a ganglion or the surrounding area. In certain embodiments, the anti-angiogenesis therapeutic is administered by intra-stellate injection, intra-DRG injection, intra-thecal injection, intra-spinal injection, epidural injection, intra-nodose injection, peri-nodose injection, intra-trigeminal ganglia injection, peri-trigeminal ganglia injection, intra-celiac ganglia injection, peri-celiac ganglia injection, intra-superior mesenteric ganglia injection, peri-superior mesenteric ganglia injection, peri-stellate administration, and peri-DRG administration. In certain embodiments, the anti-angiogenesis therapeutic is administered via intraganglionic administration such as intra-DRG, intra/peri-stellate ganglia, and intra- or peri-nodose ganglia. In certain embodiments, the anti-angiogenesis therapeutic is administrated to the nodose ganglia. In a particular embodiment, the therapeutic agents are administered via within the lumbar dorsal root ganglions (e.g., by an epidural injection). In certain embodiments, the intra-DRG injection is made at one or more of the lumbar regions L1-L6, particularly L4-L6. In certain embodiments, the intra-DRG injection is at L4 and/or L5. In certain embodiments, the anti-angiogenesis therapeutic is administered to or around a peripheral ganglion. In certain embodiments, the anti-angiogenesis therapeutic is administered to or around a dorsal root ganglion, particularly a thoracic dorsal root ganglion. In certain embodiments, the anti-angiogenesis therapeutic is administered to or around a stellate ganglion. In certain embodiments, the anti-angiogenesis therapeutic is administered intra stellate ganglion.

Injections of the instant invention may be performed using a variety of techniques including image guided or ultrasound guided injections. Injections such as intra-stellate injection may be performed using a variety of approaches including but not limited to: an ultrasound guided minimally invasive stellate ganglia block approach or other similar approaches.

In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), antioxidants (e.g., ascorbic acid, sodium metabisulfite), preservatives (e.g., thimerosal, benzyl alcohol) and bulking substances (e.g., lactose, mannitol). Common carriers include, without limitation, water, oil, buffered saline, ethanol, polyol (for example, glycerol, propylene glycol, liquid polyethylene glycol and the like), dimethyl sulfoxide (DMSO), detergents, suspending agents, glucose, lactose, gum acacia, gelatin, mannitol, starch paste, magnesium trisilicate, talc, corn starch, keratin, colloidal silica, potato starch, urea, medium chain length triglycerides, dextrans, other carriers suitable for use in manufacturing preparations, in solid, semisolid, or liquid form, and suitable mixtures thereof. In addition, excipients and auxiliary, stabilizing, preserving, thickening, flavoring, and coloring agents may be included in the compositions. The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (see, e.g., Remington's Pharmaceutical Sciences and Remington: The Science and Practice of Pharmacy). The pharmaceutical composition of the present invention can be prepared, for example, in liquid form, or can be in dried powder form (e.g., lyophilized for later reconstitution).

The therapeutic agents described herein will generally be administered to a subject/patient as a pharmaceutical preparation. The term “patient” as used herein refers to human or animal subjects. The compositions of the instant invention may be employed therapeutically or prophylactically, under the guidance of a physician. The compositions comprising the agent of the instant invention may be conveniently formulated for administration with any pharmaceutically acceptable carrier(s). The concentration of agent in the chosen medium may be varied and the medium may be chosen based on the desired route of administration of the pharmaceutical preparation. Except insofar as any conventional media or agent is incompatible with the agent to be administered, its use in the pharmaceutical preparation is contemplated. In a particular embodiment, the pharmaceutical compositions are formulated for injection into ganglion.

The dose and dosage regimen of the therapeutic agent according to the invention that is suitable for administration to a particular patient may be determined by a physician considering the patient's age, sex, weight, general medical condition, and the specific condition for which the agent is being administered to be treated or prevented and the severity thereof. The physician may also take into account the route of administration, the pharmaceutical carrier, and the agent's biological activity. Selection of a suitable pharmaceutical preparation will also depend upon the mode of administration chosen.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment or prevention therapy. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation or prevention of a particular condition may be determined by dosage concentration curve calculations, as known in the art.

The pharmaceutical preparation comprising the therapeutic agent may be administered at appropriate intervals until the pathological symptoms are reduced or alleviated, after which the dosage may be reduced to a maintenance level. The appropriate interval in a particular case would normally depend on the condition of the patient.

Toxicity and efficacy (e.g., therapeutic, preventative) of the particular formulas described herein can be determined by standard pharmaceutical procedures such as, without limitation, in vitro, in cell cultures, ex vivo, or on experimental animals. The data obtained from these studies can be used in formulating a range of dosage for use in human. The dosage may vary depending upon form and route of administration. Dosage amount and interval may be adjusted individually to levels of the active ingredient which are sufficient to deliver a therapeutically or prophylactically effective amount.

### DEFINITIONS

The following definitions are provided to facilitate an understanding of the present invention.

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition or symptom resulting in a decrease in the probability that the subject will develop the condition or symptom.

A “therapeutically effective amount” of a compound or a pharmaceutical composition refers to an amount effective to prevent, inhibit, treat, or lessen the symptoms of a particular disorder or disease. The treatment of a disease herein may refer to curing, relieving, and/or preventing a disease, the symptom(s) of it, or the predisposition towards it.

As used herein, the term “subject” refers to an animal, particularly a mammal, particularly a human.

As used herein, “diagnose” refers to detecting and identifying a disease or disorder in a subject. The term may also encompass assessing or evaluating the disease or disorder status (severity, progression, regression, stabilization, response to treatment, etc.) in a patient known to have the disease or disorder.

As used herein, the term “prognosis” refers to providing information regarding the impact of the presence of a disease or disorder (e.g., as determined by the diagnostic methods of the present invention) on a subject's future health (e.g., expected morbidity or mortality). In other words, the term “prognosis” refers to providing a prediction of the probable course and outcome of a disease/disorder or the likelihood of recovery from the disease/disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The following example describes illustrative methods of practicing the instant invention and is not intended to limit the scope of the invention in any way.

### EXAMPLE

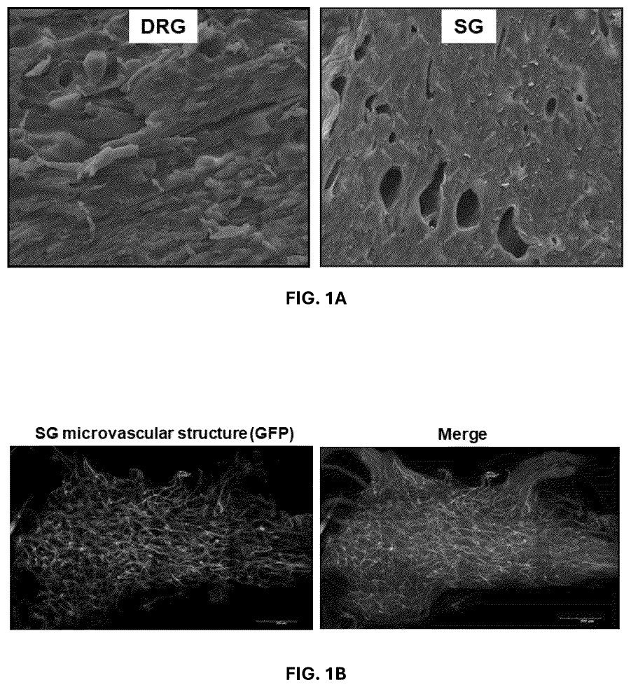

The stellate ganglia (SG) and dorsal root ganglia (DRG) contain a high density of vascular structure. As shown in FIG. 1A, scanning electron microscopy (SEM) images demonstrated that both the DRG and SG contain a high density of microvascular structure. FIG. 1B provides 3-D imaging of vascular structure inside the SG of a novel Rosa-tdTomato flox/flox::Tie2 Cre reporter mouse model after tissue clearance. These data demonstrate that disabling the SG and DRG could be achieved through blocking its vascular supply.

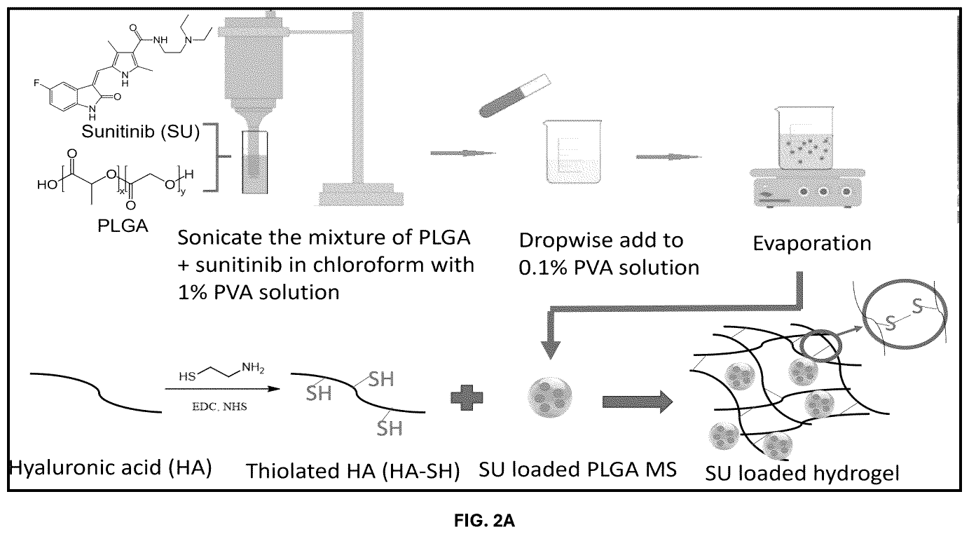

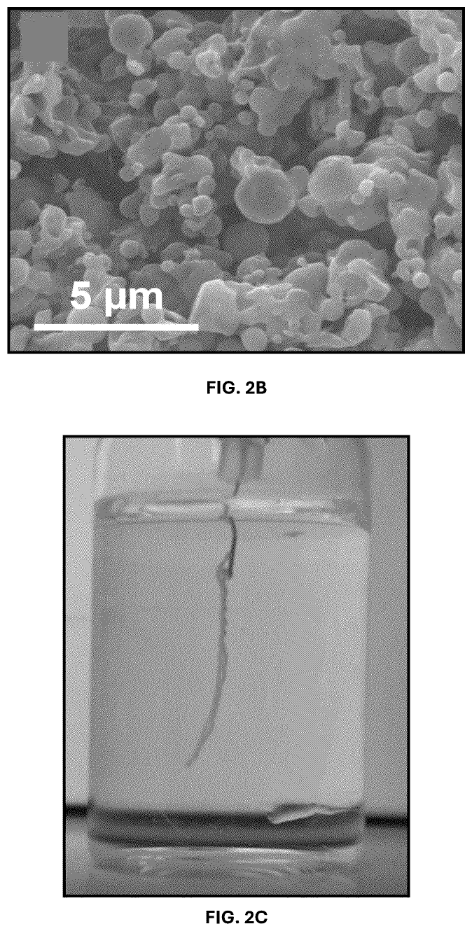

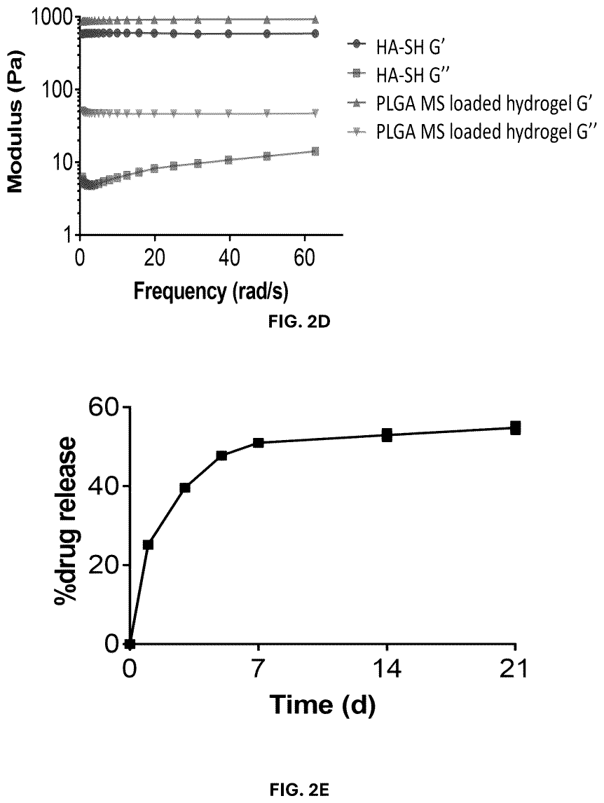

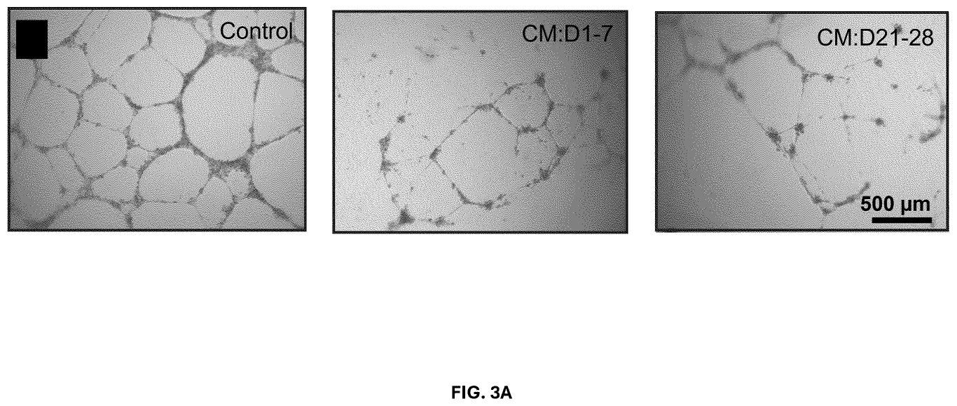

A sunitinib (SU) loaded delivery system was designed and validated in vitro. First, SU was encapsulated within PLGA microspheres by using an oil-in-water (O/W) technique (Shi et al. (2020) Carbohydr. Polym., 233:115803) (FIG. 2A). The obtained SU-PLGA microspheres had spherical morphology and had an average diameter of 1.5 μm (FIG. 2B). The SU loaded PLGA microspheres were then mixed with thiolated HA hydrogels. The hydrogels were formed due to disulfide bond formation. The SU loaded hydrogels were then injected through an insulin needle (29 G) (FIG. 2C). The hydrogels with or without PLGA microspheres had stable mechanical properties demonstrated by the frequency sweep of a rheological test (FIG. 2D). The incorporated SU was sustainedly released from PLGA microspheres and HA hydrogels for at least 21 days (FIG. 2E).

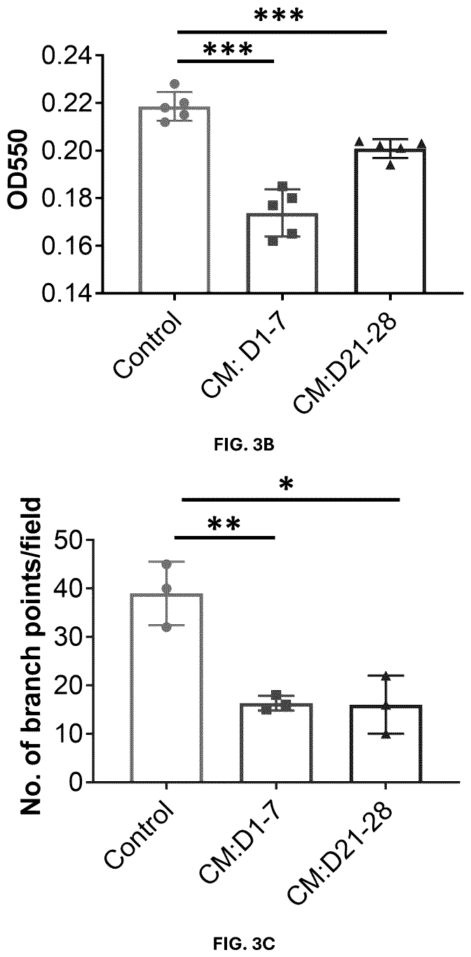

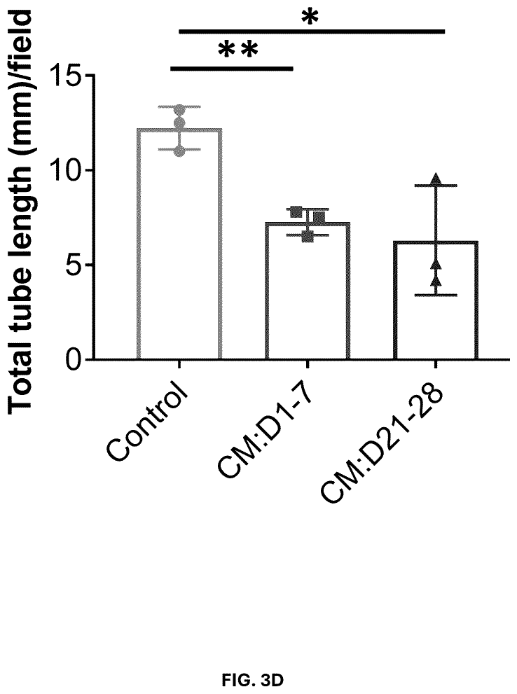

The SU loaded hydrogels was then conditioned within human umbilical vein endothelial cell (HUVEC) culture medium (CM). Culture media was then refreshed and collected every 7 days. The collected medium was then used to treat HUVECs to evaluate the cell viability and tube formation ability. The medium collected at day 1-7 and day 21-28 significantly decreased HUVEC viability (FIGS. 3A and 3B) and impaired the angiogenesis by decreasing the branch number and tube length (FIGS. 3A, 3C, and 3D). The conditioned media showed dramatic reduction in angiogenesis and vasculature.

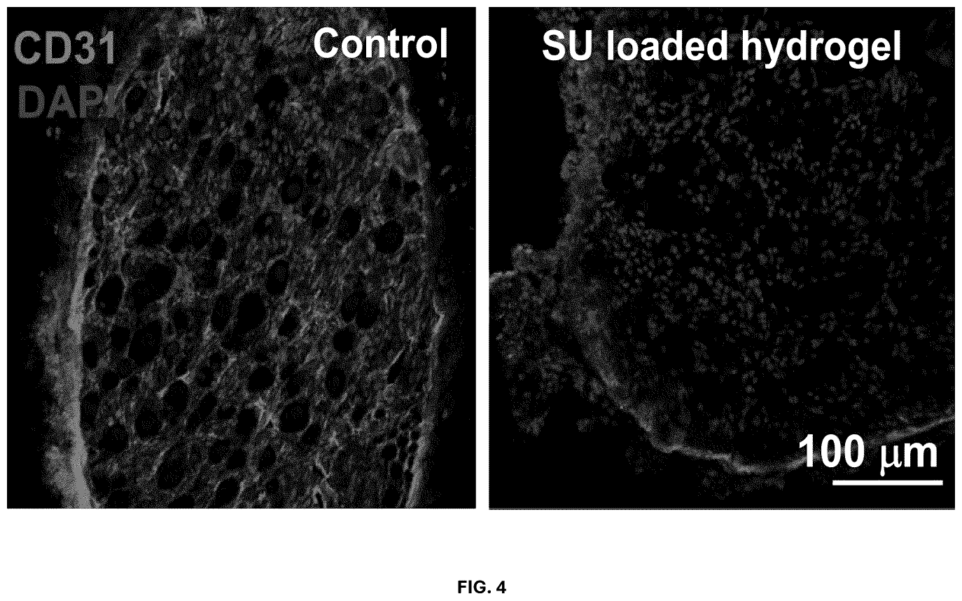

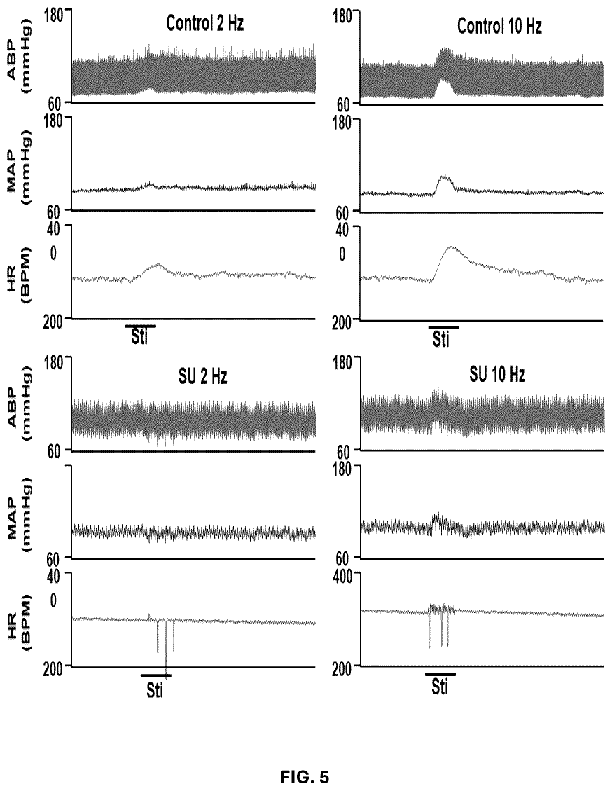

Intra-SG injection of SU loaded hydrogels was determined to disrupt SG vascular structure and ablate electrical SG stimulation-induced tachycardia and pressor responses. The SU loaded hydrogels were successfully injected into the SG (5 μl hydrogels with 10 mg PLGA microspheres containing 0.5 mg SU/each side) after exposing SG in the normal rats. The release of SU effectively disrupted the SG vascular structure as demonstrated by decreasing CD31 expressions (FIG. 4). Significantly, electrical stimulation of decentralized SG in a control (e.g., hydrogels alone) rat causes a frequency-dependent increase in heart rate (HR) and blood pressure (BP) (FIG. 5). However, such electrical stimulation-induced tachycardia and pressor responses were largely attenuated in an intra-SG SU loaded hydrogel-treated rat. This data surprisingly demonstrates that intra-SG injection of SU loaded hydrogels effectively disabled the SG function in a normal rat.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

Several publications and patent documents are cited in the foregoing specification in order to more fully describe the state of the art to which this invention pertains. The disclosure of each of these citations is incorporated by reference herein.

What is claimed is:

1. A method of inhibiting, treating, and/or preventing a disease or disorder, said method comprising administering an anti-angiogenesis therapeutic to or near a ganglion.

2. The method of claim 1, wherein said disease or disorder is caused by neural dysfunction.

3. The method of claim 1, wherein the anti-angiogenesis therapeutic is an anti-vascular endothelial growth factor (VEGF) therapeutic.

4. The method of claim 1, wherein the anti-angiogenesis therapeutic is an anti-VEGF receptor therapeutic.

5. The method of claim 1, wherein the anti-angiogenesis therapeutic is an mTOR inhibitor.

6. The method of claim 1, wherein the disease or disorder is selected from the group consisting of arrythmia, drug resistant arrythmia, heart failure, hypertension, cardiopulmonary disease, acute respiratory distress syndrome, acute lung injury, multi organ failure, and pain.

7. The method of claim 6, wherein said pain is selected from the group consisting of chronic pain, pain associated with osteoarthritis, arthritis, rheumatoid arthritis, back pain, cancer pain, pain related to peripheral artery disease, pain related to inflammatory conditions, pain associated with pancreatitis, and pain associated with colitis.

8. The method of claim 1, wherein the anti-angiogenesis therapeutic is administered by a route selected from the group consisting of intra-stellate injection, intra-dorsal root ganglion (DRG) injection, intra-thecal injection, intra-spinal injection, epidural injection, intra-nodose injection, peri-nodose injection, intra-trigeminal ganglia injection, peri-trigeminal ganglia injection, intra-celiac ganglia injection, peri-celiac ganglia injection, intra-superior mesenteric ganglia injection, peri-superior mesenteric ganglia injection, peri-stellate administration, and peri-DRG administration.

9. The method of claim 1, wherein said disease or disorder is arrhythmia.

10. The method of claim 1, wherein said disease or disorder is chronic pain.

11. The method of claim 1, wherein said disease or disorder is acute lung injury.

12. The method of claim 1, wherein the anti-angiogenesis therapeutic is administered to or near a peripheral ganglion.

13. The method of claim 1, wherein the anti-angiogenesis therapeutic is administered to or near a stellate ganglion.

14. The method of claim 4, wherein said anti-VEGF receptor therapeutic is a receptor tyrosine kinase (RTK) inhibitor.

15. The method of claim 14, wherein said RTK inhibitor is sunitinib.

16. The method of claim 1, wherein said anti-angiogenesis therapeutic is contained with a hydrogel.

17. The method of claim 1, wherein said anti-angiogenesis therapeutic is contained within a microsphere.

18. The method of claim 1, wherein said anti-angiogenesis therapeutic is contained within microspheres in a hydrogel.

19. A method of neuroablation, said method comprising administering an anti-angiogenesis therapeutic to or near a ganglion.
